(19) United States
(12) Patent Application Publication
(10) Pub. No.: US 2005/0276763 A1
(43) Pub. Date: Dec. 15, 2005

(54) Emulsion concentrate containing water-soluble and oil-soluble polymers

(75) Inventors: Svenja Pfeifer, Hamburg (DE); Claudia Mundt, Bremen (DE); Rolf Daniels, Salzgitter (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/132,821

(22) Filed: May 19, 2005

(30) Foreign Application Priority Data
May 19, 2004 (DE) 10 2004 025 357.9

(51) Int. Cl. A61K 7/42

(52) U.S. Cl. 424/59

(57) ABSTRACT

The present invention is the preparation and use of an emulsion concentrate obtainable by drying an oil-in-water emulsion comprising a lipid phase, one or more oil-soluble polymers; an aqueous phase, one or more water-soluble polymers, one or more UV photoprotective filters and optionally further cosmetic or dermatological active ingredients, auxiliaries and additives, to produce a water content of from 0.1 to 20% by weight, based on the total weight of the dried preparation.

### FIELD OF THE INVENTION

The present invention relates to a pulverulent emulsion concentrate and to processes for the preparation of an emulsion concentrate, characterized in that an oil-in-water emulsion comprising
a) a lipid phase in a concentration of from 10 to 85% by weight,
b) one or more water-soluble polymers in a total concentration of from 1 to 80% by weight,
c) one or more oil-soluble polymers in a total concentration of from 0.1 to 50% by weight, in each case based on the total weight of the preparation, and
d) one or more UV photoprotective filters and an aqueous phase, besides optionally further cosmetic or dermatological active ingredients, auxiliaries and additives, is dewatered or dried by drum drying, tunnel drying, spray-drying or freeze-drying.

The dried pulverulent emulsion concentrate can be mixed with various auxiliaries in order to form an end product which can be marketed.

### BACKGROUND OF THE INVENTION

The desire to appear beautiful and attractive is naturally rooted in man. Even if the beauty ideal has undergone changes over the course of time, the desire for a flawless outward appearance has always been the aim of humans. The condition and the appearance of the skin and of the skin appendages, i.e. of the hair and of the nails, is a significant part of a beautiful and attractive outward appearance.

The skin is the largest human organ. Among its many functions (for example temperature regulation and as a sensory organ), the barrier function, which prevents the skin (and thus ultimately the entire organism) from drying out, is probably the most important. At the same time, the skin acts as a protective device against the penetration and the absorption of exogenous substances and UV radiation. This barrier function is effected by the epidermis which, being the outermost layer, forms the actual protective sheath against the environment. Being about one tenth of the overall thickness, it is also the thinnest layer of the skin.

In order that the skin can completely fulfil its biological functions it requires regular cleansing and care. Cleansing the skin serves to remove dirt, perspiration and remains of dead skin particles which form an ideal breeding ground for pathogens and parasites of all types. Skincare products, generally creams, ointments or lotions, serve mostly to moisturize and regrease the skin. Active ingredients are often added to them which regenerate the skin and, for example, are intended to prevent and reduce its premature ageing (e.g. the appearance of wrinkles, lines).

Skincare products usually consist of emulsions. Emulsions are generally understood as meaning heterogeneous systems which consist of two liquids which are immiscible or only of limited miscibility and which are usually referred to as phases and in which one of the two liquids is dispersed in the form of very fine droplets in the other liquid. Outwardly and viewed with the naked eye, emulsions appear homogeneous.

If the two liquids are water and oil and oil droplets are present in finely dispersed form in water, this is an oil-in-water emulsion (O/W emulsion, e.g. milk). The basic character of a O/W emulsion is defined by the water. In the case of a water-in-oil emulsion (W/O emulsion, e.g. butter), the principle is reversed, the basic character here being determined by the oil.

The trend away from genteel pallor towards “healthy, sporty brown skin” has been unbroken for years. In order to attain this, people expose their skin to solar radiation since this brings about pigment formation in the sense of melanine formation. However, the ultraviolet radiation of sunlight also has a harmful effect on the skin. Besides the acute damage (sunburn), long-term damage, such as suffering from an increased risk of skin cancer in cases of excessive irradiation with light from the UVB region (wavelength: 280-320 nm), arises. Moreover, the excessive effect of UVB and UVA radiation (wavelength: 320-400 nm) leads to a weakening of the elastic and collagenous fibres of connective tissue. This leads to numerous phototoxic and photoallergic reactions and results in premature skin ageing.

To protect the skin, a number of photoprotective filters have therefore been developed which can be used in cosmetic preparations. These UVA and UVB filters are summarized in most industrialized countries in the form of positive lists such as Annex 7 of the Cosmetics Directive.

On account of their water content, conventional cosmetic or dermatological emulsions, for example sunscreen creams or lotions, have a number of disadvantages:
They have a high weight, which leads to higher energy consumption and higher costs during transportation.
They have a relatively large volume, which leads to lower transport capacities during transportation.
Particularly low viscosity emulsions are significantly more difficult to store and transport since the preparations can “leak”.
The application concentrations of the ingredients (e.g. UV filter concentration and thus UV filter performance) are already predefined for the consumer. Individual adaptation to the conditions at the site of application is no longer possible.

The drying, i.e. the removal of water, of emulsions is already known. If, however, the external phase is removed from an emulsion, then the droplets of the internal phase move closer together and coalescence is favoured. This invariably leads to breaking, i.e. phase separation, of the emulsion.

### SUMMARY OF THE INVENTION

It was therefore the object of the present invention to overcome the disadvantages of the prior art and to develop a “dry”, stable emulsion and a process for the preparation of the same.

Surprisingly, the object is achieved by a process for the preparation of an O/W emulsion comprising
a) a lipid phase in a concentration of from 10 to 85% by weight,
b) one or more water-soluble polymers in a total concentration of from 1 to 80% by weight,
c) one or more oil-soluble polymers in a total concentration of from 0.1 to 50% by weight,
in each case based on the total weight of the preparation, and
d) one or more UV photoprotective filters and
e) an aqueous phase, besides optionally further cosmetic or dermatological active ingredients, auxiliaries and additives, is dewatered or dried by drum drying, tunnel drying, spray-drying or freeze-drying.

For the purposes of the invention, dewatering or drying is understood as meaning the removal of volatile constituents from the emulsion to give a solid or wax-like product.

In this connection, it is preferred according to the invention if the lipid phase is present in the emulsion according to the invention in a concentration of from 5 to 85% by weight, particularly preferably in a concentration of from 10 to 60% by weight and very particularly preferably in a concentration of from 10 to 40% by weight, in each case based on the total weight of the preparation before drying.

It is further preferred according to the invention if one or more water-soluble polymers are present in a total concentration of from 1 to 80% by weight, particularly preferably in a concentration of from 2 to 50% by weight, and very particularly preferably in a concentration of from 2 to 25% by weight, in each case based on the total weight of the emulsion before drying.

In addition it is preferred according to the invention if the water content in the preparation after drying is less than 8% by weight and particularly preferably less than 5% by weight, in each case based on the total weight of the dried emulsion.

Furthermore, it may be advantageous according to the invention to mix the dried, pulverulent emulsion with auxiliaries, such as, for example, flow agents, thickeners, water-soluble or water-dispersible additives.

It is surprising and unforeseeable by the person skilled in the art that the dry emulsions can be “re-emulsified” (diluted) by simply adding water, i.e. that a liquid O/W emulsion is again formed, which is usually not the case for “dried” emulsions.

Y. Kawashima et al [Int. J. Pharmazeut., 86 (1992) 25-33, Drug Development and Industrial Pharmacy, 18(9), 919-937 (1992) and Chem. Pharm. Bull. 39(6) 1528-1531 (1991)] and H. G. Kristensen et al. [Euro. J. Pharmaceutics and Biopharmaceutics 53 (2002) 147-153 und Int. J. Pharmaceutics 212 (2001) 187-194, 195-202] describe dried redispersible emulsions. These emulsions are also stabilized using hydroxypropylmethylcellulose (water-soluble polymer). However, the emulsions described therein are nowhere near as complex as cosmetic or dermatological emulsions. For this reason, these specifications were unable to point the way to the present invention.

In the preparation according to the invention, the coalescence of the oil droplets known from the prior art is prevented by the synergistic effect of water-soluble and oil-soluble polymers. The oil-soluble polymer, e.g. ethylcellulose, stabilizes the oil droplet from within. The water-soluble polymers, e.g. hydroxypropylmethylcellulose, act as “spacers” in the water phase which remains after drying; these prevent the oil droplets from contacting with one another.

Compared with the redispersible emulsions known hitherto, the emulsions according to the invention have a particularly pleasant feel on the skin and can be combined with a large number of cosmetic active ingredients, auxiliaries and additives. For example, the emulsions according to the invention comprise UV photoprotective filters which hitherto have not been able to be incorporated into re-emulsifiable emulsions.

### DETAILED DESCRIPTION OF THE INVENTION

The oil phase of the emulsion according to the invention, i.e. the lipophilic organic constituents, is advantageously chosen from the group of polar oils, for example from the group of lecithins and of fatty acid triglycerides, namely the triglycerol esters of saturated and unsaturated, branched and unbranched alkanecarboxylic acids of chain length from 8 to 24, in particular 12 to 18, carbon atoms. The fatty acid triglycerides can, for example, be chosen advantageously from the group of synthetic, semisynthetic and natural oils, such as, for example, caprylic/capric triglycerides, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like. Also advantageous according to the invention are, for example, natural waxes of animal and vegetable origin, such as, for example, beeswax and other insect waxes, and berry wax, shea butter and lanolin (wool wax).

For the purposes of the present invention, further advantageous polar oil components can also be chosen from the group of esters of saturated and unsaturated, branched and unbranched alkanecarboxylic acids of chain length from 3 to 30 carbon atoms and saturated and unsaturated, branched and unbranched alcohols of chain length from 3 to 30 carbon atoms, and from the group of esters of aromatic carboxylic acids and saturated and unsaturated, branched and unbranched alcohols of chain length from 3 to 30 carbon atoms. Such ester oils can then be chosen advantageously from the group consisting of octyl palmitate, octyl cocoate, octyl isostearate, octyldodeceyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can be chosen advantageously from the group of dialkyl ethers and dialkyl carbonates, advantageous examples being dicaprylyl ether (Cetiol OE) and dicaprylyl carbonate, for example that are available under the trade name Cetiol CC from Cognis.

It is also preferred to choose the oil component or the oil components from the group consisting of isoeicosane, neopentyl glycol diheptanoate, propylene glycol dicaprylate/dicaprate, caprylic/capric/diglyceryl succinate, butylene glycol dicaprylate/dicaprate, cocoglycerides (e.g. MYRITOL® 331 from Henkel), C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, dimethyl isosorbide. It is particularly advantageous if the oil phase of the formulations according to the invention has a content of C₁₂₋₁₅-alkyl benzoate, or consists entirely of this. Advantageous oil components are also, for example, butyloctyl salicylate (for example that available under the trade name Hallbrite BHB from CP Hall), hexadecyl benzoate and butyloctyl benzoate and mixtures thereof (Hallstar AB) and diethylhexyl naphthalate (CORAPAN®TQ from Haarmann & Reimer). Any mixtures of such oil and wax components can also be used advantageously for the purposes of the present invention.

According to the invention, the lipid phase can comprise the polar oil components in a concentration of up to 80% by weight, based on the total weight of the lipid phase. The weight data is based here on the composition of the preparation before drying.

In addition, the oil phase can likewise advantageously also comprise nonpolar oils, for example those which are chosen from the group of branched and unbranched hydrocarbons and hydrocarbon waxes, in particular mineral oil, vaseline (petrolatum), paraffin oil, squalane and squalene, polyolefins, hydrogenated polyisobutenes and isohexadecane. Among the polyolefins, polydecenes and hydrogenated polyisobutenes are the preferred substances.

The nonpolar oil components can advantageously be present in the emulsions according to the invention in a concentration of up to 80% by weight, based on the total weight of the lipid phase. The weight data is based here on the composition of the preparation before drying.

The oil phase can also advantageously have a content of cyclic or linear silicone oils, or consist entirely of such oils, although it is preferred to use an additional content of other oil phase components apart from the silicone oil or the silicone oils. Silicone oils are high molecular weight synthetic polymeric compounds in which silicon atoms are joined in a catenated or reticular manner via oxygen atoms and the remaining valencies of the silicon are saturated by hydrocarbon radicals (in most cases methyl groups, more rarely ethyl, propyl, phenyl groups, etc.). Systematically, the silicone oils are referred to as polyorganosiloxanes. The methyl-substituted polyorganosiloxanes, which represent the most important compounds of this group in terms of amount and are characterized by the following structural formula I 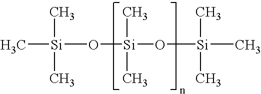
are also referred to as polydimethylsiloxane or dimethicone (INCI). Dimethicones come in various chain lengths and with various molecular weights.

For the purposes of the present invention, particularly advantageous polyorganosiloxanes are, for example, dimethylpolysiloxanes [poly(dimethylsiloxane)], which are available, for example, under the trade name Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are also referred to in accordance with INCI as Cyclomethicone, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxane (Stearoxy Dimethicone and Behenoxy Stearyl Dimethicone), which are available as various Abil-wax grades from Th. Goldschmidt. However, other silicone oils are also to be used advantageously for the purposes of the present invention, for example cetyldimethicone, hexamethylcyclotrisiloxane, polydimethylsiloxane, poly(methylphenylsiloxane). According to the invention, particularly preferred silicones are dimethicone and cyclomethicone.

The fraction of silicone oil in the lipid phase can advantageously be from 20 to 100% by weight and particularly preferably from 30 to 60% by weight, in each case based on the total weight of the lipid phase.

Water-soluble polymers advantageous according to the invention can be chosen advantageously according to the invention from the group of water-soluble or dispersible film formers (e.g. polyurethanes, dimethicone copolyol polyacrylate, polyvinylpyrrolidone-vinyl acetate PVP/VA, polyvinylpyrrolidone (PVP), polyvinyl alcohols (e.g. Mowiol 18-88) and from the group of hydrocolloids (e.g. agar agar, carrageen, tragacanth, gum arabic, alginates, pectins, polyoses, guar flour, carob seed grain, starch, dextrins, polysaccharide N-alkylurethanes, inulin carbamates, gelatin, casein, cellulose ethers, hydroxyethylcellulose and hydroxypropylcellulose derivatives, polysaccharides, polyacrylic and polymethacrylic compounds, ammonium acryloyl dimethyltaurate/vinylpyrrolidone copolymers and ammonium polyacryldimethyltauramides, vinyl polymers, polycarboxylic acids, polyethers, polyimines, polyamides, polysilicic acids, clay minerals, zeolites, silicas, see below).

Hydrocolloids preferred according to the invention are, for example, methylcelluloses, which is the term used for the methyl ethers of cellulose. They are characterized by the following structural formula II 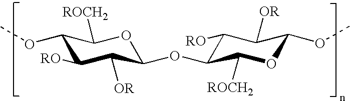
in which R may be a hydrogen or a methyl group.

In particular, for the purposes of the present invention, the cellulose mixed ethers, generally likewise referred to as methylcelluloses and which additionally comprise 2-hydroxyethyl, 2-hydroxypropyl or 2-hydroxybutyl groups besides a dominant content of methyl groups, are advantageous. Particularly preferred hydroxypropylmethylcelluloses (HPMCs) have an average molar mass Mₘ<50 000 gmol⁻¹ and are available, for example, under the name Pharmacoat 603, Pharmacoat 606 and Pharmacoat 645 or under the name Metolose 65 SH 50 or Metolose 60 SH 50 from Shin Etsu. Further preferred hydroxypropylmethylcelluloses are available under the trade name Methocel K100LV and Methocel E4M from Dow Chemicals or under the trade name Methocel E5 from Colorcon. Also advantageous according to the invention is sodium carboxymethylcellulose, the sodium salt of the glycolic acid ether of cellulose, for which R in structural formula II may be a hydrogen or CH2—COONa. The sodium carboxymethylcellulose obtainable under the trade name Natrosol Plus 330 CS from Aqualon and also referred to as cellulose gum is particularly preferred.

In addition, it is in accordance with the invention if such an O/W emulsion according to the invention comprises water-soluble cosmetic or dermatological active ingredients, auxiliaries, additives, or combinations thereof.

For the purposes of the present invention, oil-soluble polymers advantageous according to the invention can be chosen from the group of organomodified bentonites (e.g. Bentone 38 V from Nordmann and Rassmann) and from the group of hydrophobically modified bentonites (e.g. Tixogel VP-V from Südchemie), and from the group of highly disperse silicon dioxides (Aerosils). Advantageous substances are also ethylene/propylene/styrene copolymer, butylene/ethylene/styrene copolymer (e.g. VERSAGEL® from Penreco) and hydrogenated polydecene, ethylene/propylene/styrene copolymer, butylene/ethylene/styrene copolymer (e.g. DEKAGEL® from Jan Dekker).

Also advantageous according to the invention are oil-soluble, structure-imparting substances, such as allyl methacrylate crosspolymer (e.g. POLY-PORE L 200® from Chemdal Corporation), silicone polyamide (e.g. DOW CORNING 2-8178® from Dow Corning), dextrin palmitate, polyurethanes and trihydroxystearin. Oil-soluble polymers particularly preferred according to the invention may be chosen from the group of cellulose ethers, e.g. ethylcellulose (abbreviation according to DIN 7728-1:1988-01: EC), in particular ETHOCEL® from Dow Chemical Company.

According to the invention, the use of one or more oil-soluble polymers in a total concentration of from 0.1 to 50% by weight, in particular in a concentration of from 0.5 to 10% by weight, is preferred.

When using a combination of oil-soluble and water-soluble polymers it has surprisingly been found that, as a result of synergistic interplay, the absolute concentration of the ingredients necessary for preventing phase separation, in particular of water-soluble and oil-soluble polymers, can be reduced.

A further advantage which is achieved through the combination of oil-soluble and water-soluble polymers is the relatively high oil content in the dried emulsion relative to the polymer concentration and the associated improvement of sensory properties.

A not insignificant secondary effect is that, as result of the addition of the oil-soluble polymer, the water resistance of the redispersed emulsion on the skin is considerably increased compared to the redispersed emulsion which comprises only water-soluble polymers.

The invention also provides the process for the preparation of an O/W emulsion according to the invention which is characterized in that a pulverulent O/W emulsion according to the invention dried by drum drying, tunnel drying, spray-drying, freeze-drying, or combinations thereof is mixed with one or more water-soluble or readily volatile active ingredients, auxiliaries, or additives in a mixing device.

According to the invention, “readily volatile” means that these compounds have a boiling point of at most 100° C. and would thus may be removed azeotropically or by fractionation in the drying process. An O/W emulsion which is prepared by such a process is in accordance with the invention.

According to the invention, the emulsion according to the invention can thus comprise water-soluble and water-dispersible ingredients. According to the invention, it may be advantageous to add these after drying.

According to the invention, the emulsion according to the invention can comprise water-soluble ingredients, for example alcohols, diols or polyols of low carbon number, and ethers thereof, preferably ethanol, isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous substances. The emulsions can comprise one or more polyols chosen from the group consisting of sorbitol, mannitol, propylene glycol and butylene glycol. The polyols particularly preferred according to the invention are sorbitol and mannitol. It is advantageous for the purposes of the present invention if the cosmetic or dermatological emulsion according to the invention comprises a total amount of polyols of from 5.0 to 40.0% by weight, preferably from 7.5 to 35.0% by weight and very particularly preferably from 10.0 to 25.0% by weight, in each case based on the total weight of the preparation. The weight data refer here to the composition of the preparation before drying.

It is in accordance with the invention that the consumer of the dried emulsion adds a certain amount of water or another liquid and, after the action of motive forces, such as, for example, shaking or stirring, a redispersed emulsion is obtained. The temperature of the added water or of the other liquid is between 20 and 110° C. in accordance with the invention. The redispersed emulsion according to the invention is advantageously used as sunscreen composition.

It is also advantageous for the purposes of the present invention to provide cosmetic and dermatological preparations whose main purpose is not protection against sunlight but which nevertheless comprise a content of UV protection substances. Thus, for example, UV-A and/or UV-B filter substances are usually incorporated into day creams or makeup products. UV protection substances, like antioxidants and, if desired, preservatives, also represent an effective protection of the preparations themselves against spoilage.

It is likewise in accordance with the invention that the consumer uses the pulverulent emulsion concentrate as “body powder” directly, without redispersion.

Accordingly, the preparations for the purposes of the present invention preferably comprise at least one UV-A or UV-B filter substance. The formulations may, but not necessarily, optionally also comprise one or more organic or inorganic pigments as UV filter substances.

Preferred inorganic pigments are metal oxides or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, and the sulphate of barium (BaSO₄).

The titanium dioxide pigments may be present either in the rutile or anatase crystal modification and, for the purposes of the present invention, may advantageously be surface-treated (“coated”), the intention being, for example, to form or retain a hydrophilic, amphiphilic or hydrophobic character. This surface treatment can consist in providing the pigments with a thin hydrophilic or hydrophobic inorganic or organic layer by processes known per se. For the purposes of the present invention, the different surface coatings can also comprise water. For the purposes of the present invention, described coated and uncoated titanium dioxides can also be used in the form of commercially obtainable oily or aqueous predispersions. Dispersion auxiliaries and solubilization promoters can advantageously be added to these predispersions. The titanium dioxides according to the invention are characterized by a primary particle size between 10 nm to 150 nm and are available under the following trade names from the companies listed:

| | | Additional | |
|---|---|---|---|
| | | constituents | |
| | | of the | |
| Trade name | Coating | predispersion | Manufacturer |
| MT-100TV | Aluminium hydroxide | — | Tayca |
| | Stearic acid | | Corporation |
| MT-100Z | Aluminium hydroxide | — | Tayca |
| | Stearic acid | | Corporation |
| MT-100F | Stearic acid | — | Tayca |
| | Iron oxide | | Corporation |
| MT-500SAS | Alumina, silica | — | Tayca |
| | Silicone | | Corporation |
| MT-100AQ | Silica | — | Tayca |
| | Aluminium hydroxide | | Corporation |
| | Alginic acid | | |
| Eusolex T-2000 | Alumina | — | Merck KgaA |
| | Simethicone | | |
| Eosolex TS | Alumina, stearic acid | — | Merck KgaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titan X170 | Alumina | — | Kemira |
| | Dimethicone | | |
| UV-Titan X161 | Alumina, silica | — | Kemira |
| | Stearic acid | | |
| Tioveil AQ | Alumina | Water | Solaveil |
| 10PG | Silica | Propylene | Uniqema |
| | | glycol | |
| Mirasun TiW 60 | Alumina | Water | Rhone-Poulenc |
| | Silica | | |

For the purposes of the present invention, particularly preferred titanium dioxides are the MT-100 Z and MT-100 TV from Tayca Corporation, Eusolex T-2000 and Eusolex TS from Merck and the titanium dioxide T 805 from Degussa.

For the purposes of the present invention, zinc oxides can also be used in the form of commercially available oily or aqueous predispersions. Zinc oxide particles and predispersions of zinc oxide particles suitable according to the invention are characterized by a primary particle size of <300 nm and are available under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |
| MZ 707M | 7% Dimethicone | M. Tayca Corp. |
| Nanox 500 | / | Elementis |
| ZnO Neutral | / | H&R |

Particularly preferred zinc oxides for the purposes of the invention are Z-Cote HP1 from BASF and the zinc oxide NDM from Haarmann & Reimer.

The total amount of one or more inorganic pigments in the finished cosmetic preparation is advantageously chosen from the 0.1% by weight to 25% by weight range, preferably 0.5% by weight to 18% by weight, based on the preparation before drying.

An advantageous organic pigment for the purposes of the present invention is 2,2′-methylenebis(6-(2H-benzotriazol-2-yl)-4-(1,1,3,3-tetramethylbutyl)phenol) [INCI: Bisoctyltriazole], which is available under the trade name TINOSORB® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the brand PARSOL® 1789 and by Merck under the tradename EUSOLEX® 9020.

Advantageous further UV filter substances for the purposes of the present invention are sulphonated, water-soluble UV filters, such as, for example:
phenylene-1,4-bis(2-benzimidazyl)-3,3′-5,5′-tetrasulphonic acid and its salts, particularly the corresponding sodium, potassium or triethanolammonium salts, in particular the phenylene-1,4-bis(2-benzimidazyl)-3,3′-5,5′-tetrasulphonic acid bis-sodium salt with the INCI name Disodium Phenyl Dibenzimidazole Tetrasulphonate (CAS No.: 180898-37-7), which is available, for example, under the trade name Neo Heliopan AP from Haarmann & Reimer;
salts of 2-phenylbenzimidazole-5-sulphonic acid, such as its sodium, potassium or triethanolammonium salt, and the sulphonic acid itself with the INCI name Phenylbenzimidazole Sulphonic Acid (CAS No. 27503-81-7), which is available, for example, under the trade name Eusolex 232 from Merck or under Neo Heliopan Hydro from Haarmann & Reimer;
1,4-di(2-oxo-10-sulpho-3-bornylidenemethyl)benzene (also: 3,3′-(1,4-phenylenedimethylene)bis(7,7-dimethyl-2-oxobicyclo[2.2.1]hept-1 -ylmethanesulphonic acid) and salts thereof (particularly the corresponding 10-sulphato compounds, in particular the corresponding sodium, potassium or triethanolammonium salt), which is also referred to as benzene-1,4-di(2-oxo-3-bornylidenemethyl-10-sulphonic acid). Benzene-1,4-di(2-oxo-3-bornylidenemethyl-10-sulphonic acid) has the INCI name Terephthalidene Dicamphor Sulphonic Acid (CAS No.: 90457-82-2) and is available, for example, under the trade name Mexoryl SX from Chimex;
sulphonic acid derivatives of 3-benzylidenecamphor, such as, for example 4-(2-oxo-3-bornylidenemethyl)benzenesulphonic acid, 2-methyl-5-(2-oxo-3-bornylidenemethyl)sulphonic acid and salts thereof.

Advantageous UV filter substances for the purposes of the present invention are also benzoxazole derivatives which are characterized by the following structural formula III, 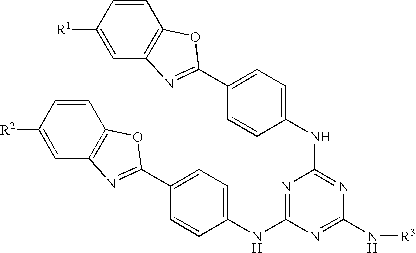
in which R¹, R² and R³, independently of one another, are chosen from the group of branched and unbranched, saturated and unsaturated alkyl radicals having 1 to 10 carbon atoms. According to the invention, it is particularly advantageous to choose the radicals R¹ and R² to be the same, in particular from the group of branched alkyl radicals having 3 to 5 carbon atoms. It is also particularly advantageous for the purposes of the present invention if R³ is an unbranched or branched alkyl radical having 8 carbon atoms, in particular the 2-ethylhexyl radical.

A benzoxazole derivative which is particularly preferred according to the invention is 2,4-bis[5-1(dimethylpropyl)benzoxazol-2-yl(4-phenyl)imino]-6-(2-ethylhexyl)imino-1,3,5-triazine with the CAS No. 288254-16-0 which is characterized by the structural formula IV 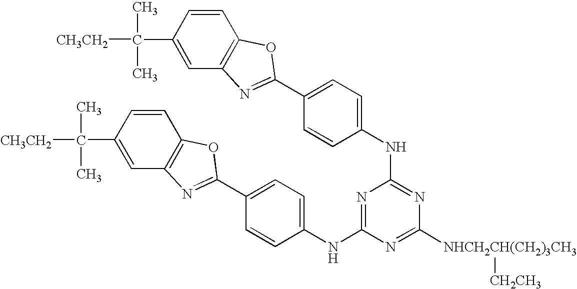
and is available from 3V Sigma under the trade name UVASORB® K2A.

The benzoxazole derivative or derivatives are advantageously in dissolved form in the cosmetic preparations according to the invention. It may, however, also be advantageous in some circumstances if the benzoxazole derivative or derivatives are present in pigmentary, i.e. undissolved form—for example in particle sizes of from 10 nm to 300 nm.

Advantageous UV filter substances for the purposes of the present invention are also so-called hydroxybenzophenones. Hydroxybenzophenones are characterized by the following structural formula V: 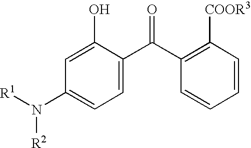
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5- or 6-membered ring and
R³ is a C₁-C₂₀-alkyl radical.

A particularly advantageous hydroxybenzophenone for the purposes of the present invention is hexyl 2-(4′-diethylamino-2′-hydoxybenzoyl)benzoate (also: aminobenzophenone) which is characterized by the following structure VI: 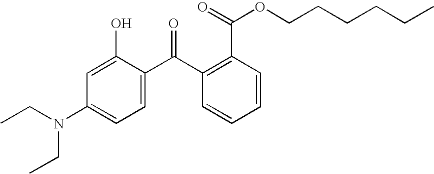
and is available under the trade name Uvinul A Plus from BASF.

Advantageous UV filters substances for the purposes of the present invention are also so-called broadband filters, i.e. filter substances which absorb both UV-A and UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,5-triazine (INCI: Bis-Ethylhexyloxylphenol Methoxyphenyl Triazine), which is available under the trade name TINOSORB® S from CIBA-Chemikalien GmbH;
dioctylbutylamidotriazone (INCI: Diethylhexyl Butamido Triazone), which is available under the trade name UVASORB HEB from Sigma 3V;
tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150;
2-[4,6-bis(2,4-dimethylphenyl)-1,3,5-triazin-2-yl]-5-(octyloxy)phenol (CAS No.: 2725-22-6).

An advantageous broadband filter for the purposes of the present invention is also 2,2′-methylenebis(6-(2H-benzotriazol-2-yl)-4-(1,1,3,3-tetramethylbutyl)phenol) (INCI: Methylene Bis-Benztriazolyl Tetramethylbutylphenol), which is available, for example, under the trade name TINOSORB® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The further UV filter substances may be oil-soluble or water-soluble. Advantageous oil-soluble filter substances are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino(p-carbo-2′-ethyl-1′-hexyloxy)-1,3,5-triazine;
esters of benzyl malonic acid, preferably di(2-ethylhexyl) 4-methoxybenzylmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone; and
UV filters bonded to polymers.

Advantageous water-soluble filter substances are, for example: sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and salts thereof.

A further photoprotective filter substance to be used advantageously according to the invention is ethylhexyl 2-cyano-3,3-diphenylacrylate, which is available from BASF under the name UVINUL® N 539 T.

Besides the filter substance(s) according to the invention, particularly advantageous preparations for the purposes of the present invention which are characterized by high or very high UV-A protection preferably also comprise further UV-A or broadband filters, in particular dibenzoylmethane derivatives [for example 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine and/or phenylene-1,4-bis(2-benzimidazyl)-3,3′-5,5′-tetrasulphonic acid bis-sodium salt, in each case individually or in any combinations with one another. The list of specified UV filters which can be used for the purposes of the present invention is not of course intended to be limiting.

The preparations according to the invention advantageously comprise the substances which absorb UV radiation in the UV-A or UV-B region in a total amount of, for example, 0.1% by weight to 40% by weight, preferably 0.5 to 30% by weight, in particular 1.0 to 20% by weight, in each case based on the total weight of the preparations, in order to make available cosmetic preparations which protect the hair and/or the skin from the entire range of ultraviolet radiation. They can also serve as sunscreens for the hair or the skin. The weight data here refer to the composition of the preparation before drying.

Particularly preferred embodiments of the present invention comprise, as UV filters, one or more triazine derivatives, dibenzoylmethane derivatives, UV filters liquid at room temperature, or inorganic pigments, in particular titanium dioxide.

Furthermore, it is in accordance with the invention that the user uses the dried emulsion directly, e.g. as “body powder” without the addition of further water.

In addition, it may in some instances be advantageous to incorporate film formers into the emulsion according to the invention, for example in order to improve the water resistance of the preparations or to increase the UV protection performance (UV-A and/or UV-B boosting). Both water-soluble or dispersible and fat-soluble film formers are suitable, in each case individually or in combination with one another.

Water-soluble or dispersible film formers advantageous according to the invention which can also be used as water-soluble polymers according to the invention are, for example, polyurethanes (e.g. the AVALURE® grades from Goodrich), dimethicone copolyol polyacrylate (Silsoft SURFACE® from the Witco Organo Silicones Group), PVPNA (VA=vinyl acetate) copolymer (Luviscol VA 64 Powder from BASF), polyvinyl alcohols etc.

Advantageous water-soluble film formers are, for example, the film formers from the group of polymers based on polyvinylpyrrolidone (PVP), having the structure VII:

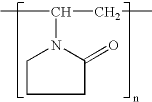

Particular preference is given to copolymers of polyvinylpyrrolidone, for example the PVP hexadecene copolymer and the PVP eicosene copolymer, which are available under the trade names Antaron V216 and Antaron V220 from GAF Chemicals Cooperation, and Tricontayl PVP and the like.

According to the invention, the customary antioxidants can be used in the emulsion. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacectic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of these specified active ingredients.

The amount of the antioxidants (one or more compounds) in the preparations is preferably 0.001 to 30% by weight, particularly preferably 0.025-6.0% by weight, in particular 0.05-3.0% by weight, based on the total weight of the preparation. The weight data refer here to the composition of the preparation before drying.

If vitamin A or vitamin A derivatives, or carotenes or derivatives thereof are the antioxidant or the antioxidants, it is advantageous to choose their respective concentrations from the range from 0.001 to 30% by weight, based on the total weight of the formulation. The weight data here refer to the composition of the preparation before drying.

If vitamin E and/or derivatives thereof are the antioxidant or the antioxidants, it is advantageous to choose their respective concentrations from the range from 0.001 to 30% by weight, based on the total weight of the formulation. The weight data here refer to the composition of the preparation before drying.

Further advantageous active ingredients for the purposes of the present invention are natural active ingredients and derivatives thereof, such as, for example, alpha-lipoic acid, phytoene, D-biotin, coenzyme Q10, alpha-glucosylrutin, carnitine, carnosine, natural and/or synthetic isoflavonoids, creatine, fumaric esters, ectoin and derivatives thereof, taurine, and β-alanine. Based on the total weight of the emulsion, these active ingredients may be present therein in a concentration of from 0.001 to 30% by weight. The weight data here refer to the composition of the preparation before drying.

Formulations according to the invention which comprise, for example, known antiwrinkle active ingredients, such as flavone glycosides (in particular α-glycosylrutin), coenzyme Q10, vitamin E and derivatives and the like are particularly advantageously suitable for the prophylaxis and treatment of cosmetic or dermatological changes in the skin, as arise, for example, during skin ageing (such as, for example, dryness, roughness and formation of dryness wrinkles, itching, reduced regreasing (e.g. after washing), visible vascular dilations (telangiectasias, couperosis), flaccidity and formation of lines and wrinkles, local hyperpigmentation, hypopigmentation and incorrect pigmentation (e.g. age spots), increased susceptibility to mechanical stress (e.g. cracking) and the like). In addition, they are advantageously suitable for combating the appearance of dry and/or rough skin.

However, other pharmaceutically or dermatologically effective substances, such as, for example, substances which calm and care for the skin, can also be incorporated into the preparations according to the invention. These include, for example, panthenol, allantoin, tannin, antihistamines (e.g. loratadine, cetirizine, dimethiondene, clemastine, capsaicin, H₁ antagonists, tannin preparations), local anaesthetics, opiate antagonists (e.g. naltrexone, naloxone), antiphlogistics, glucocorticoids (e.g. hydrocortisone, tacrolimus, ciclosporin A) and plant active ingredients, such as azulene and bisabolol, glycyrrhizin, hamamelin and plant extracts, such as camomile, aloe vera, hamamelis, liquorice (Glycyrhiza glabra and inflata). The vitamin D₃ analogues tacalcitol, calcipotriol, colecalciferol and calcitrol (vitamin D₃) and/or fumaric esters can also be successfully incorporated into the preparations.

Based on the total weight of the emulsion, these active ingredients may be present therein in a concentration of from 0.001 to 30% by weight. The weight data here refer to the composition of the preparation before drying.

Advantageous moisturizing and humectant agents (so-called moisturizers) for the purposes of the present invention are, for example, glycerol, lactic acid and/or lactates, in particular sodium lactate, butylene glycol, propylene glycol, biosaccaride gum-1, glycine soya, ethylhexyloxyglycerol, pyrrolidonecarboxylic acid and urea. In addition, it is particularly advantageous to use polymeric moisturizers from the group of water-soluble, or water-swellable, or water-gellable polysaccharides. Of particular advantage are, for example, hyaluronic acid, chitosan, or a fucose-rich polysaccharide, which is deposited in Chemical Abstracts under the registry number 178463-23-5 and is available, for example, under the name FUCOGEL®1000 from SOLABIA S.A.

The preparations according to the invention can advantageously comprise solids carriers in the form of microfine solid particles. According to the invention, these can advantageously be surface-treated (“coated”) to repel water, the intention being to form or retain an amphiphilic character of these solid particles. The surface treatment can consist in providing the solid particles with a thin hydrophobic or hydrophilic layer by methods known per se.

The average particle diameter of the microfine solids carriers used as stabilizer is preferably chosen to be less than 100 μm, particularly advantageously less than 50 μm. Here, it is essentially unimportant in which form (platelets, rods, beads, etc.) or modification of the solid particles used are present.

The microfine solids carriers are preferably chosen from the group of amphiphilic metal oxide pigments. In particular, the following are advantageous:
titanium dioxides (coated and uncoated): e.g. Eusolex T-2000 from Merck, titanium dioxide MT-100 Z from Tayca Corporation
zinc oxide e.g. Z-Cote and Z-Cote HP1 from BASF AG, MZ-300, MZ-500 and MZ-505M from Tayca Corporation
iron oxides
   Furthermore, it is advantageous if the microfine solids carriers are chosen from the following group: boron nitrides, starch derivatives (tapioca starch, sodium maize starch octenylsuccinate etc.), talc, latex particles.

According to the invention, the microfine solid particles are advantageously used in a concentration of from 0.5 to 60% by weight, preferably in a concentration of from 1 to 50% by weight and particularly preferably in a concentration of from 3 to 30% by weight, in each case based on the total weight of the preparation. The weight data here refer to the composition of the preparation before drying.

The cosmetic or dermatological preparations according to the invention may also advantageously, but not necessarily, comprise fillers which, for example, further improve the sensory and cosmetic properties of the formulations and bring about or enhance a velvety or silky feel on the skin. Advantageous fillers for the purposes of the present invention are starch and starch derivatives (such as, for example, tapioca starch, distarch phosphate, aluminium or sodium starch octenylsuccinate and the like), pigments which have neither primarily a UV filter effect nor coloring effect (such as, for example, boron nitride etc.) and/or AEROSILS® (CAS No. 7631-86-9).

Apart from the abovementioned substances, according to the invention, the compositions optionally comprise the additives customary in cosmetics, for example perfume, dyes, antimicrobial substances, regreasing agents, complexing and sequestering agents, pearlescent agents, plant extracts, vitamins, active ingredients, preservatives, bactericides, repellents, self-tanning agents (e.g. DHA), depigmenting agents (e.g. 8-hexadecene-1,16-dicarboxylic acid (dioic acid, CAS Number 20701-68-2; provisional INCI name Octadecenedioic acid)), pigments which have a coloring effect, softening, moisturizing and/or humectant substances, or other customary constituents of a cosmetic or dermatological formulation, such as emulsifiers, polymers, foam stabilizers, peeling substances (abrasives, e.g. polymer beads or powders made of polyethylene, polypropylene etc. inorganic oxides, silicates etc.), antiperspirant salts (e.g. acidic aluminium and/or aluminium/zirconium salts, such as aluminium chlorohydrate and/or aluminium/zirconium chlorohydrate) and electrolytes.

According to the invention, the repellent active ingredients listed below are preferred:

| | | | Effectiveness |
|---|---|---|---|
| | | | (literature and |
| Chemical | Trade | | manufacturer |
| name | name | Structure | details) |
| butopyronoxyl | Indalone | 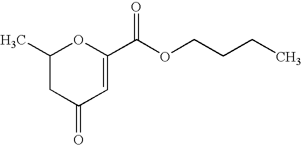 | biting insects¹ |
| 2,3;4,5-bis(2- butylene)tetra- hydro-2- furaldehyde | MGK- Repellent 11 | 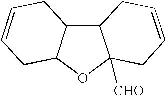 | cockroaches and biting insects¹ |
| N,N- diethylcapryl- amide | Repellent 790 | 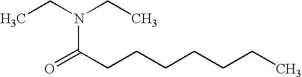 | cockroaches, mosquitoes, houseflies, horseflies, ants, arachnids, |
| o-chloro-N,N-di- ethylbenzamide in a mixture with N,N-diethyl- benzamide | Kik- Repellent | 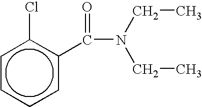 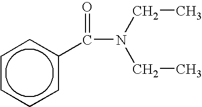 | mosquitoes, horseflies, fleas, bugs, ticks, flies, lice |
| dimethyl carbate | Dimalone | 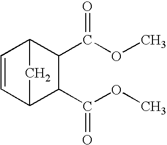 | mosquitoes, in particular of the Aedes species¹ |
| di-n-propyl iso- cinchomeronate | MGK- Repellent 326 | 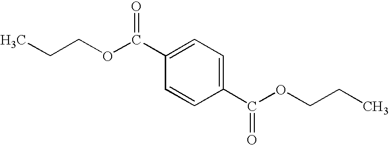 | houseflies, bush flies¹ |
| 2-ethylhexane- 1,3-diol | Rutgers 612 | 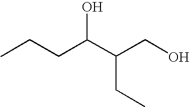 | mosquitoes, horseflies, flies, fleas, mites¹ |
| N-octylbicyclo- heptenedicarbox- imide | MGK 264 Insecticide- synergist | 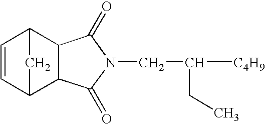 | synergist² |
| piperonyl butoxide | PBO | 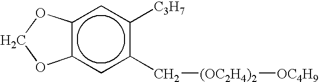 | synergist² |

¹predominantly in a mixture or combination with other repellents
²acts as synergist with various repellents

Particularly advantageous repellent active ingredients for the purposes of the present invention are the abovementioned active ingredients N,N-diethyl-3-methylbenzamide, ethyl 3-(N-n-butyl-N-acetylamino)propionate and dimethyl phthalate. Very particular preference is given to the repellent ethyl 3-(N-n-butyl-N-acetylamino)propionate.

Embodiments of the emulsion according to the invention which are advantageous according to the invention comprise one or more repellent active ingredients in a concentration of 1-50% by weight, based on the total weight of the formulation. The weight data here refer to the composition of the preparation before drying.

Self-tanning agents which can be used advantageously according to the invention are, inter alia: glycerol aldehyde, hydroxymethylglyoxal, γ-dialdehyde, erythrulose, 6-aldo-D-fructose, ninhydrin, 5-hydroxy-1,4-naphthoquinone (Juglon), 2-hydroxy-1,4-naphthoquinone (Lawson) and particularly preferably 1,3-dihydroxyacetone.

Embodiments advantageous according to the invention with at least one self-tanning substance comprise these in a total concentration of from 0.1 to 30% by weight, based on the total weight of the emulsion. The weight data here refer to the composition of the preparation before drying.

According to the invention, the emulsion according to the invention can advantageously comprise one or more preservatives. Advantageous preservatives for the purposes of the present invention are, for example, formaldehyde donors (such as, for example, DMDM hydantoin, which is available, for example, under the trade name GLYDANT™ from Lonza), iodopropyl butylcarbamates (e.g. those available under the trade names Glycacil-L, Glycacil-S from Lonza and/or Dekaben LMB from Jan Dekker), parabens (i.e. alkyl p-hydroxybenzoates, such as methyl, ethyl, propyl and/or butyl paraben), phenoxyethanol, ethanol, benzoic acid and the like. In addition, according to the invention, the preservatives system usually also advantageously comprises preserving aids, such as, for example, octoxyglycerol, glycine soya etc. The table below gives an overview of some preservatives which are advantageous according to the invention:

| | |
|---|---|
| E 200 | sorbic acid |
| E 201 | sodium sorbate |
| E 202 | potassium sorbate |
| E 203 | calcium sorbate |
| E 210 | benzoic acid |
| E 211 | sodium benzoate |
| E 212 | potassium benzoate |
| E 213 | calcium benzoate |
| E 214 | ethyl p-hydroxybenzoate |
| E 215 | ethyl p-hydroxybenzoate Na salt |
| E 216 | n-propyl p-hydroxybenzoate |
| E 217 | n-propyl p-hydroxybenzoate Na salt |
| E 218 | methyl p-hydroxybenzoate |
| E 219 | methyl p-hydroxybenzoate Na salt |
| E 220 | sulphur dioxide |
| E 221 | sodium sulphite |
| E 222 | sodium hydrogensulphite |
| E 223 | sodium disulphite |
| E 224 | potassium disulphite |
| E 226 | calcium sulphite |
| E 227 | calcium hydrogensulphite |
| E 228 | potassium |
| | hydrogensulphite |
| E 230 | biphenyl (diphenyl) |
| E 231 | orthophenylphenol |
| E 232 | sodium |
| | orthophenylphenoxide |
| E 233 | thiabendazole |
| E 235 | natamycin |
| E 236 | formic acid |
| E 237 | sodium formate |
| E 238 | calcium formate |
| E 239 | hexamethylenetetramine |
| E 249 | potassium nitrite |
| E 250 | sodium nitrite |
| E 251 | sodium nitrate |
| E 252 | potassium nitrate |
| E 280 | propionic acid |
| E 281 | sodium propionate |
| E 282 | calcium propionate |
| E 283 | potassium propionate |
| E 290 | carbon dioxide |

Also advantageous are preservatives or preserving auxiliaries customary in cosmetics, such as dibromodicyanobutane (2-bromo-2-bromomethylglutaronitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates. According to the invention, it is particularly preferred if the preservatives used are iodopropyl butylcarbamates, parabens (methyl, ethyl, propyl and/or butyl paraben) and/or phenoxyethanol.

According to the invention, one or more preservatives are advantageously present in a concentration of from 0.001 to 2% by weight, preferably 0.01 to 1.5% by weight and particularly preferably 0.05 to 1% by weight, in each case based on the total weight of the preparation. The weight data here refer to the composition of the preparation before drying.

The emulsion according to the invention advantageously comprises one or more conditioners. Conditioners preferred according to the invention are, for example, all compounds which are listed in the International Cosmetic Ingredient Dictionary and Handbook (Volume 4, editors: R. C. Pepe, J. A. Wenninger, G. N. McEwen, The Cosmetic, Toiletry, and Fragrance Association, 9th edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all of the compounds listed in EP 0934956 (pp. 11-13) under water soluble conditioning agent and oil soluble conditioning agent. Some of these compounds are listed by name under the constituents of the aqueous phase and of the oil phase. Further conditioners advantageous according to the invention are, for example, the compounds named Polyquaternium according to the international nomenclature for cosmetic ingredients (INCI) (in particular Polyquaternium-1 to Polyquaternium-56).

According to the invention, the emulsion according to the invention can advantageously comprise glitter substances and other effect substances.

Hydrocolloids advantageous according to the invention that may be used as water-soluble polymers according to the invention are agar agar, carrageen, tragacanth, gum arabic, alginates, pectins, polyoses, guar flour, carob seed grain, starch, dextrins, polysaccharide N-alkylurethanes, inulin carbamates, gelatin, casein, cellulose ethers, hydroxyethylcellulose and hydroxypropylcellulose derivatives, polysaccharides, polyacrylic and polymethacrylic compounds, ammonium acryloyldimethyltaurates/vinylpyrrolidone copolymers and ammonium polyacryloyldimethyltauramides, vinyl polymers, polycarboxylic acids, polyethers, polyimines, polyamides, polysilicic acids, clay minerals, zeolites, and silicas.

An advantageous hydrocolloid for the purposes of the present invention is also xanthan (CAS No. 11138-66-2), also called xanthan gum, which is an anionic heteropolysaccharide, which is usually formed by fermentation from maize sugar and is isolated as the potassium salt.

An advantageous hydrocolloid for the purposes of the present invention is also carrageen, a gel-forming extract from North Atlantic red algae—belonging to the Florideae (Chondrus crispus and Gigartina stellata) which has a similar structure to agar.

Polyacrylates are hydrocolloids likewise to be used advantageously for the purposes of the present invention. Polyacrylates advantageous according to the invention are acrylate-alkyl acrylate copolymers, in particular those which are chosen from the group of so-called carbomers or carbopols (CARBOPOL® is actually a registered trade mark of NOVEON Inc.). In particular, the acrylate-alkyl acrylate copolymers advantageous according to the invention are characterized by the following structure VIII:

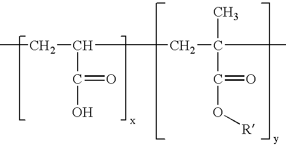

In this structure, R′ is a long-chain alkyl radical and x and y are numbers which symbolize the respective stoichiometric fraction of the particular comonomers.

According to the invention, preference is given to acrylate copolymers and/or acrylate-alkyl acrylate copolymers which are available under the trade names CARBOPOL® 1382, CARBOPOL® 981 and CARBOPOL® 5984, Aqua SF-1 from NOVEON Inc. and as ACULYN® 33 from International Specialty Products Corp. Further preference is given to the carbomers Carbopol EDT 2001, ETD 2020 and ETD 2050.

Also advantageous are copolymers of C10-30-alkyl acrylates and one or more monomers of acrylic acid, of methacrylic acid or esters thereof which are crosslinked with an allyl ether of sucrose or an allyl ether of pentaerythritol.

Compounds which bear the INCI name “Acrylates/C 10-30 Alkyl Acrylate Crosspolymer” are advantageous. Those available under the trade names PEMULEN® TR1 AND PEMULEN® TR2 from NOVEON Inc. are particularly advantageous.

Compounds which bear the INCI name “AcrylatesNinyl Isodecanoate Crosspolymer” are advantageous. Those available under the trade names Stabylen 30 from 3V Sigma are particularly advantageous.

Also advantageous are compounds which have the INCI name “acrylates/C12-24 pareth-25 acrylate copolymer” (available under the trade names Synthalen® W2000 from 3V Inc.), which have the INCI name “acrylates/steareth-20 methacrylate copolymer” (available under the trade names Aculyn® 22 from the International Specialty Products Corp.), which have the INCI name “acrylates/steareth-20 itaconate copolymer” (available under the trade names STRUCTURE 2001® from National Starch), which have the INCI name “acrylates/aminoacrylates/C10-30 alkyl PEG-20 itaconate copolymer” (available under the trade names Structure Plus® from National Starch) and similar polymers. According to the invention, it is particularly preferred to use neutralized or partially neutralized polyacrylates (e.g. Carbopols from Noveon).

The cosmetic and/or dermatological emulsions according to the invention can comprise a number of pigments. The dyes and color pigments can be chosen from the corresponding positive list of cosmetics legislation or the EC list of cosmetic colorants. In most cases, they are identical to the dyes approved for foods. Advantageous color pigments are, for example, titanium dioxide, mica, iron oxides (e.g. Fe₂O₃, Fe₃O₄, FeO(OH)) or tin oxide. Advantageous dyes are, for example, carmine, Prussian blue, chromium oxide green, ultramarine blue and manganese violet. It is particularly advantageous to choose the dyes and color pigments from the following list. The Color Index numbers (CIN) can be found in the Rowe Color Index, 3rd edition, Society of Dyers and Colorists, Bradford, England, 1971.

| Chemical or other name | CIN | Color |
|---|---|---|
| Pigment Green | 10006 | green |
| Acid Green 1 | 10020 | green |
| 2,4-dinitrohydroxynaphthalene-7-sulphonic acid | 10316 | yellow |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-chloro-4′-nitro-1′-phenylazo)-2- | 12085 | red |
| hydroxynaphthalene | | |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-methoxy-5′-sulphonic acid diethylamide-1′- | 12490 | red |
| phenylazo)-3-hydroxy-5″-chloro-2″,4″-dimethoxy-2- | | |
| naphthoic acid anilide | | |
| Disperse Yellow 16 | 12700 | yellow |
| 1-(4-sulpho-1-phenylazo)-4-aminobenzene-5-sulphonic | 13015 | yellow |
| acid | | |
| 2,4-dihydroxyazobenzene-4′-sulphonic acid | 14270 | orange |
| 2-(2,4-dimethylphenylazo-5-sulphonic acid)-1- | 14700 | red |
| hydroxynaphthalene-4-sulphonic acid | | |
| 2-(4-sulpho-1-naphthylazo)-1-naphthol-4- | 14720 | red |
| sulphonic acid | | |
| 2-(6-sulpho-2,4-xylylazo)-1-naphthol-5-sulphonic acid | 14815 | red |
| 1-(4′-sulphophenylazo)-2-hydroxynaphthalene | 15510 | orange |
| 1-(2-sulphonic acid-4-chloro-5-carboxylic acid-1- | 15525 | red |
| phenylazo)-2-hydroxynaphthalene | | |
| 1-(3-methylphenylazo-4-sulphonic acid)-2- | 15580 | red |
| hydroxynaphthalene | | |
| 1-(4′,(8′)-sulphonic acid-naphthylazo)-2- | 15620 | red |
| hydroxynaphthalene | | |
| 2-hydroxy-1,2′-azonaphthalene-1′-sulphonic acid | 15630 | red |
| 3-hydroxy-4-phenylazo-2-naphthylcarboxylic acid | 15800 | red |
| 1-(2-sulpho-4-methyl-1-phenylazo)-2- | 15850 | red |
| naphthylcarboxylic acid | | |
| 1-(2-sulpho-4-methyl-5-chloro-1-phenylazo)- | 15865 | red |
| 2-hydroxy-naphthalene-3-carboxylic acid | | |
| 1-(2-sulpho-1-naphthylazo)-2-hydroxynaphthalene-3- | 15880 | red |
| carboxylic acid | | |
| 1-(3-sulpho-1-phenylazo)-2-naphthol-6-sulphonic acid | 15980 | orange |
| 1-(4-sulpho-1-phenylazo)-2-naphthol-6-sulphonic acid | 15985 | yellow |
| Allura Red | 16035 | red |
| 1-(4-sulpho-1-naphthylazo)-2-naphthol-3,6-disulphonic | 16185 | red |
| acid | | |
| Acid Orange 10 | 16230 | orange |
| 1-(4-sulpho-1-naphthylazo)-2-naphthol-6,8-disulphonic | 16255 | red |
| acid | | |
| 1-(4-sulpho-1-naphthylazo)-2-naphthol- | 16290 | red |
| 3,6,8-trisulphonicacid | | |
| 8-amino-2-phenylazo-1-naphthol-3,6-disulphonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-sulpho-1-phenylazo)-1-(4-sulphophenyl)- | 19140 | yellow |
| 5-hydroxy-pyrazolone-3-carboxylic acid | | |
| Pigment Yellow 16 | 20040 | yellow |
| 2,6-(4′-sulpho-2″,4″-dimethyl)bisphenylazo)-1,3-di- | 20170 | orange |
| hydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-sulpho-1″-phenylazo)-7′- | 27755 | black |
| sulpho-1′-naphthylazo]-1-hydroxy-7- | | |
| aminonaphthalene-3,6-disulphonic acid | | |
| 4′-[(4″-sulpho-1″-phenylazo)-7′- | 28440 | black |
| sulpho-1′-naphthylazo]-1-hydroxy-8- | | |
| acetylaminonaphthalene-3,5- | | |
| disulphonic acid | | |
| Direct Orange 34, 39, 44, 46, 60 | 40215 | orange |
| Food Yellow | 40800 | orange |
| trans-β-apo-8′-carotenealdehyde (C₃₀) | 40820 | orange |
| trans-apo-8′-carotenic acid (C₃₀) ethyl ester | 40825 | orange |
| Canthaxanthin | 40850 | orange |
| Acid Blue 1 | 42045 | blue |
| 2,4-disulpho-5-hydroxy-4′-4″- | 42051 | blue |
| bis(diethylamino)triphenyl-carbinol | | |
| 4-[(4-N-ethyl-p-sulphobenzylamino)phenyl- | 42053 | green |
| (4-hydroxy-2-sulphophenyl)(methylene)-1- | | |
| (N-ethyl-N-p-sulphobenzyl)-2,5-cyclohexadieneimine] | | |
| Acid Blue 7 | 42080 | blue |
| (N-ethyl-p-sulphobenzylamino)phenyl- | 42090 | blue |
| (2-sulphophenyl)-methylene(N-ethyl-N- | | |
| p-sulphobenzyl)Δ^{2.5}- | | |
| cyclohexadieneimine | | |
| Acid Green 9 | 42100 | green |
| diethyldisulphobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-methyl-4′-(N-ethyl-N-m-sulphobenzyl)amino-4″-(N- | 42735 | blue |
| diethyl)-amino-2-methyl-N-ethyl-N-m- | | |
| sulphobenzylfuchsonimmonium | | |
| 4′-(N-dimethyl)amino-4″-(N-phenyl)aminonaphtho-N- | 44045 | blue |
| dimethyl-fuchsonimmonium | | |
| 2-hydroxy-3,6-disulpho-4,4′-bisdimethylamino- | 44090 | green |
| naphthofuchsonimmonium | | |
| Acid Red 52 | 45100 | red |
| 3-(2′-methylphenylamino)-6-(2′-methyl-4′-sulpho- | 45190 | violet |
| phenylamino)-9-(2″-carboxyphenyl)xanthenium salt | | |
| Acid Red 50 | 45220 | red |
| phenyl-2-oxyfluorone-2-carboxylic acid | 45350 | yellow |
| 4,5-dibromofluorescein | 45370 | orange |
| 2,4,5,7-tetrabromofluorescein | 45380 | red |
| Solvent Dye | 45396 | orange |
| Acid Red 98 | 45405 | red |
| 3′,4′,5′,6′-tetrachloro-2,4,5,7-tetrabromofluorescein | 45410 | red |
| 4,5-diiodofluorescein | 45425 | red |
| 2,4,5,7-tetraiodofluorescein | 45430 | red |
| quinophthalone | 47000 | yellow |
| quinophthalonedisulphonic acid | 47005 | yellow |

It may also be favourable to choose as the dye one or more substances from the following group: 2,4-dihydroxyazobenzene, 1-(2′-chloro-4′-nitro-1′-phenylazo)-2-hydroxynaphthalene, Ceres red, 2-(4-sulpho-1-naphthylazo)-1-naphthol-4-sulphonic acid, calcium salt of 2-hydroxy-1,2′-azonaphthalene-1′-sulphonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminium salt of 1-(4-sulpho-1-phenylazo)-2-naphthol-6-sulphonic acid, aluminium salt of 1-(4-sulpho-1-naphthylazo)-2-naphthol-3,6-disulphonic acid, 1-(4-sulpho-1 -naphthylazo)-2-naphthol-6,8-disulphonic acid, aluminium salt of 8-amino-2-phenylazo-1-naphthol-3,6-disulphonic acid, aluminium salt of 4-(4-sulpho-1-phenylazo)-1-(4-sulphophenyl)-5-hydroxy-pyrazolone-3-carboxylic acid, 4′-[(4″-sulpho-1″-phenylazo)-7′-sulpho-1′-naphthylazo]-1-hydroxy-8-acetylaminonaphthalene-3,5-disulphonic acid, aluminium and zirconium salts of 4,5-dibromofluorescein, aluminium and zirconium salts of 2,4,5,7-tetrabromofluoroscein, 3′,4′,5′,6′-tetrachloro-2,4,5,7-tetrabromofluorescein and its aluminium salt, aluminium salt of 2,4,5,7-tetraiodofluorescein, aluminium salt of quinophthalone-disulphonic acid, aluminium salt of indigodisulphonic acid, 4,4′-dimethyl-6,6′-dichlorothioindigo, complex salt (Na, Al, Ca) of carminic acid, red and black iron oxide (CIN: 77 491 (red) and 77 499 (black)), iron oxide hydrate (CIN: 77 492), manganese ammonium diphosphate (CIN 77745), ultramarine (CIN 77007) and titanium dioxide.

Emulsions according to the invention can comprise titanium dioxides, which may be present either in the rutile or the anatase crystal modification and, for the purposes of the present invention, is advantageously surface-treated (“coated”), the intention being, for example, to form or retain a hydrophilic, amphiphilic or hydrophobic character. This surface treatment can consist in providing the pigments with a thin hydrophilic or hydrophobic inorganic and organic layer by processes known per se. The various surface coatings can also comprise water for the purposes of the present invention.

Inorganic surface coatings for the purposes of the present invention can consist of aluminium oxide (Al₂O₃), aluminium hydroxide Al(OH)₃, or aluminium oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), zirconium oxide (ZrO₂) or iron oxide (Fe₂O₃). These inorganic surface coatings may be present on their own, in combination and/or in combination with organic coating materials.

For this purpose, oxides, oxide hydrates or phosphates, for example of the elements Al, Si, Zr, are precipitated on the pigment surface in dense layers.

The inorganic after-treatment generally takes place in an aqueous suspension of the pigment by adding soluble after-treatment chemicals, such as, for example, aluminium sulphate, and subsequent precipitation of the hydroxide, which is sparingly soluble in the neutral range, by controlled adjustment of the pH with sodium hydroxide solution.

After the inorganic after-treatment, the coated pigments are separated off from the suspension by filtration and washed carefully in order to remove the dissolved salts, and the isolated pigments are then dried.

Of particular preference for the purposes of this invention are titanium dioxides onto whose surface aluminium hydroxide has been applied, such as, for example, the titanium dioxide grades C47-051 and C47-5175 obtainable from Sun Chemical. Further preferred pigments are titanium dioxides which are coated with aluminium oxides and/or silicon oxides, such as, for example, from Krosnos Titan: Kronos 1071 and 1075 or from Kingfisher: A310.03 Tudor Aspen.

Organic surface coatings for the purposes of the present invention can consist of vegetable or animal aluminium stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

In addition, it may be advantageous according to the invention to use pearlescent pigments.

These include natural pearlescent pigments, such as, for example,
“fish silver” (guanine/hypoxanthine mixed crystals from fish scales) and
“mother-of-pearl” (ground mussel shells),
   monocrystalline pearlescent pigments, such as, for example, bismuth oxychloride (BiOCl), layer-substrate pigments: e.g. mica/metal oxide

The bases of pearlescent pigments are, for example, pulverulent pigments or castor oil dispersions of bismuth oxychloride and/or titanium dioxide, and bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| Group | thickness | Color |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | golden |
| | | shades |
| | TiO₂/Cr₂O₃ | green |
| | TiO₂/Prussian blue | deep blue |
| | TiO₂/carmine | red |

Particular preference is given to the pearlescent pigments obtainable from Merck under the trade names Timiron, Colorona or Dichrona.

The list of specified pearlescent pigments is not of course intended to be limiting. Pearlescent pigments advantageous for the purposes of the present invention are obtainable by numerous routes known per se. For example, substrates other than mica can be coated with further metal oxides, such as, for example, silica and the like. SiO₂ particles coated, for example, with TiO₂ and Fe₂O₃ (“Ronaspheres”), which are sold by Merck, are advantageous.

It may, moreover, be advantageous to completely dispense with a substrate such as mica. Particular preference is given to pearlescent pigments which are prepared using SiO₂. Such pigments, which can also additionally have goniochromatic effects, are obtainable, for example, under the trade name Sicopearl Fantastico from BASF.

Pigments from Engelhard/Mearl which are based on calcium sodium borosilicate and which are coated with titanium dioxide can also be used advantageously. These are available under the name Reflecks. Due to their particle size of 40-180 μm, they have a glitter effect in addition to the color.

The dyes and pigments can be present either individually or else in a mixture, and can also be mutually coated with one another, various color effects in general being brought about by different coating thicknesses. The total amount of the dyes and coloring pigments is advantageously chosen from the range from, for example, 0.1% by weight to 30% by weight, preferably from 0.5 to 15% by weight, in particular from 1.0 to 10% by weight, in each case based on the total weight of the preparations. The weight data here refer to the composition of the preparation before drying.

For the purposes of the present invention, the O/W emulsion according to the invention advantageously comprises one or more tablet auxiliaries. These can be used according to the invention advantageously in a concentration of from 0.1 to 60% by weight, preferably in a concentration of from 0.1 to 50% by weight and very particularly preferably in a concentration of from 0.5 to 35% by weight, in each case based on the total weight of the preparation. The weight data here refer to the composition of the preparation, the preparation being regarded as being the emulsion after drying together with any further water-soluble and/or readily volatile compounds and tabletting substances (including hydrogencarbonates and acids solid at room temperature).

Tablet auxiliaries which may be used are, for example, filling and compacting auxiliaries (e.g. starch derivatives and/or cellulose derivatives), flow agents (e.g. highly disperse silicon dioxides), flow regulators, lubricants and mould release agents. These tablet auxiliaries can expressly also be added if the product is left as powder and not further processed to give a tablet. Particularly preferred flow agents are obtainable, for example, under the name Sipernat from Degussa.

According to the invention, all substances can advantageously be used which are listed in H. P. Fielder, Lexikon der Hilfsstoffe für Pharmazie, Kosmetik und angrenzenden Gebiete [Lexicon of Auxiliaries for Pharmacy, Cosmetics and Related Fields], 5th edition, Editio Cantor Verlag, Aulendorf, 2002, under the keywords granulation auxiliaries, tablet binders, tablet fillers, tablet glidants, tablet auxiliaries, tablet disintegrants, tablet coatings.

In an embodiment which is particularly preferred according to the invention, the preparation according to the invention comprises a combination of carbonates and hydrogencarbonates, and acids solid at room temperature (e.g. citric acid, ascorbic acid, lactic acid, tartaric acid, etc). Strongly swelling polymers, such as, for example, crosslinked polyvinylpyrrolidone, can also be used advantageously according to the invention.

According to the invention, one or more hydrogencarbonates (e.g. sodium hydrogencarbonate, potassium hydrogencarbonate) are advantageously used in an amount of from 0.1 to 30% by weight, preferably in an amount of from 0.5 to 20% by weight and particularly preferably in an amount of from 0.5 to 15% by weight, in each case based on the total weight of the preparation, the preparation being regarded as being the emulsion after drying together with any further water-soluble and/or readily volatile compounds and tabletting substances (including hydrogencarbonates and acids that are solid at room temperature).

According to the invention, one or more acids solid at room temperature (particularly preferably citric acid) are advantageously used in an amount of from 0.1 to 30% by weight, preferably in an amount of from 0.5 to 20% by weight and particularly preferably in an amount of from 0.5 to 15% by weight, in each case based on the total weight of the preparation, the preparation being regarded as being the emulsion after drying together with any further water-soluble or readily volatile compounds and tabletting substances (including hydrogen carbonates and acids solid at room temperature).

Such an embodiment is especially advantageous for the purposes of the present invention if the emulsion according to the invention is in the form of a tablet (e.g. an effervescent tablet) since the emulsion according to the invention in this embodiment can be re-emulsified particularly readily by adding water.

The invention also provides the process for producing such a preparation, which is characterized in that the O/W emulsion according to the invention is combined with one or more tablet auxiliaries in accordance with the modular principle and mixed in a mixing device. This process of mixing the dried emulsion with auxiliaries is understood for the purposes of the present invention as meaning mixing to give the product which can be marketed. The mixing aims to produce a product which is stable, flowable depending on the area of use, pressable into tablets and/or storage-stable. In particular, the addition, i.e. the mixing, of readily volatile substances, such as, for example, perfume substances, is only possible after drying. It is also occasionally necessary to add release agents which achieve the flowability. The case of spray-drying in particular, voluminous products are formed whose bulk density can be increased by adding tabletting auxiliaries.

The invention also provides preparations which are prepared by this process according to the invention. The invention also provides a process for the preparation of tablets or granules which is characterized in that a preparation according to the invention is pressed or granulated in a press to give one or more tablets, and also preparations which are prepared by this process.

Last but not least, the invention provides the process for the preparation of a cosmetic which is characterized in that a preparation according to the invention (a dried O/W emulsion which has optionally been admixed with water-soluble and/or readily volatile active ingredients, auxiliaries, additives and/or tablet auxiliaries, and has optionally been tabletted and/or granulated) is admixed with water or emulsified in water. The invention also provides cosmetics which are prepared by this process.

During spray-drying, the material to be dried (liquid solution, suspension or emulsion) is sprayed at the upper end of a wide, cylindrical container through nozzles or by means of a rapidly rotating atomizer disc to give a fine mist. Hot air or an inert gas (in the case of oxidation-sensitive substances) is passed from below countercurrently to the resulting spray cone. In the case of very temperature-sensitive products (e.g. enzymes, active microorganisms) the introduction of the drying gas in cocurrent from above is recommended. The large relative surface of the liquid droplets brings about an effective and rapid exchange of heat and material between the two phases. The dry material drops down as a more or less fine powder, as granules or in the form of small beads (so-called prills) and is discharged at the bottom of the dryer by mechanical rakers, conveyor screws or the like. Dust particles which remain in the exit air have to be obtained by centrifugal force separators (cyclones). The capacity of industrial spray dryers can reach 100 t/h of steam. It is an aim of process technological optimization of spray drying to arrange the feed rate of gas and heat such that it corresponds to the evaporation rate of the water from the droplets.

Freeze-drying (lyophilization) is understood as meaning a basic operation of processing technology in which the liquid phase (in most cases water) is removed by sublimation from the frozen material while circumventing the liquid state of aggregation.

It is clear from the phase diagram for water that sublimation can take place only below the triple point. For freeze-drying, therefore, the temperature at which a solid phase is only just present (eutectic range) is important. For products with a complex composition this point is in most cases much lower than for water and the water vapour partial pressure is very low at the sublimation surface. For this reason, a corresponding vacuum is required for the freeze-drying. During the drying operation, the temperature of the material to be dried must not exceed this value in order to prevent thawing.

The energy required for the sublimation (2845 kJ/kg of water) has to be introduced from outside to the sublimation zone during the drying operation. This occurs through contact, through radiation and, depending on the vacuum in the drying chamber, through convection. Since below 10⁻² mbar heat exchange is virtually no longer possible by convection, the vacuum must always be kept as bad as possible, but still good enough to prevent thawing. The pressure must therefore be regulated very precisely during freeze-drying.

Freeze-drying is certainly the gentlest form of drying and is used for bioproducts which are particularly temperature-sensitive. Freeze-drying takes place mainly in vacuum tunnel dryers or vacuum plate dryers over the course of 1 to 3 hours. The advantages of freeze-drying are the low temperatures at which no chemical change in the product arises. However, the technical complexity (generation of the vacuum and of the low temperature of −45° C. in the condenser) and the specific energy requirement are high, meaning that it has hitherto been used on cost-intensive products, such as hormone, enzyme and vitamin preparations, plasma and serum banks for blood transfusion, viruses, bacteria (live vaccines, strain maintenance).

During tunnel drying, the spread-out wet material is mechanically conveyed (conveyor belt) to pass through a tunnel which is heated by steam, hot water or hot air. Ventilators ensure air circulation. The dry material is removed at the end of the tunnel. Drying drums and those devices in which the drying material is conveyed by stirrers or other internals, e.g. screw-conveyor dryer, paddle dryer or trough dryer, are also advantageous. They are likewise heated and permit a continuous flow of material.

Cylinder dryers have proven useful for removing the liquid from viscous material. From a storage container, the solvent is applied in a thin layer to a metal cylinder heated from within using a doctor roller, and contact drying takes place. The drying process lasts only a few seconds then after brief rotation the dried material is removed from the cylinder using scrapers. Substances which cannot withstand even this short-term heating can be dewatered on vacuum cylinder dryers.

Such a cosmetic according to the invention is then, after drying and subsequent renewed rehydration, in the form of an ointment, cream, lotion or an emulsion foam or a sprayable form. According to the invention, it can be used advantageously for the treatment and care of the skin, hair and nails. According to the invention, preference is given here to the use as sunscreen compositions.

It is also in accordance with the invention to apply the dried emulsion directly to the skin without the further addition of water, i.e. without redispersion. This can take place in the form of, for example, “body powders” or sticks, in particular powder sticks and/or concealing sticks.

The examples below are intended to illustrate the present invention without limiting it. Unless stated otherwise, all of the amounts, fractions and percentages given are based on the weight and the total amount or on the total weight of the preparations.

The example preparations 1 to 5 (emulsion preparations before drying) can, in accordance with the invention, be homogeneously mixed with auxiliary mixtures 1 to 5 (comprising water-soluble/dispersible additives and/or tabletting and granulation auxiliaries) in accordance with the “modular principle”. The mixing ratio of dried emulsion and auxiliary mixture is advantageously chosen to be in the range 1000:1 to 1:2.

### EXAMPLES

Preparation 1: Photoprotective preparation

| | % by wt. |
|---|---|
| Ethylcellulose | 0.7 |
| Hydroxypropylmethylcellulose | 25 |
| Titanium dioxide (Eusolex T2000) | 6 |
| Nylon-12 | 1 |
| Hydrogenated coco glycerides | 2 |
| Caprylic/capric triglyceride | 10 |
| Octyldodecanol | 10 |
| Mineral oil | 3 |
| Butylene glycol caprylate/caprate | 7 |
| C₁₂₋₁₅-alkyl benzoate | 6 |
| Cyclomethicone | 2 |
| Starch hydroxypropyltrimonium chloride | 1 |
| (Sensomer CI 50) | |
| Bisethylhexyloxyphenol methoxyphenyl | 4 |
| triazine | |
| Ethylhexyl methoxycinnamate | 8 |
| Octocrylene | 6 |
| Preservative | 2 |
| Glycerol | 5 |
| Water-soluble dye | 0.3 |
| Water | ad 100 |

Preparation 2: O/W self-tanning emulsion

| | % by wt. |
|---|---|
| Ethylcellulose | 0.5 |
| Ethylene/propylene/styrene/copolymer | 0.5 |
| Hydroxypropylmethylcellulose | 30 |
| Xanthan gum | 2 |
| C₁₀₋₃₀-Alkyl acrylate cross polymer | 1 |
| (Pemulen TR1) | |
| Boron nitride | 2 |
| Distarch phosphate | 1 |
| Polyamide-6 | 1 |
| Hydrogenated coco glycerides | 2 |
| Cetyldimethicone (Abil Wax 9840) | 2 |
| Caprylic/capric triglyceride | 10 |
| Octyldodecanol | 10 |
| Dicaprylyl carbonate | 5 |
| Stearyl alcohol | 2 |
| Dimethicone | 2 |
| PPG-15 stearyl ether | 1 |
| Hydrogenated polyisobutene | 1 |
| (Polysynlan) | |
| Dihydroxyacetone | 4 |
| Fat-soluble dye | 1 |
| Distarch phosphate | 2 |
| Glycerol | 7 |
| Panthenol | 3 |
| Sorbitol | 5 |
| Lactic acid | 1 |
| Hydroxyethylcellulose | 1 |
| Polyacrylate | 2 |
| Water | ad 100 |

Preparation 3: Photoprotective preparation

| | % by wt. |
|---|---|
| Ethylcellulose | 2 |
| Quaternium-90 bentonite | 0.5 |
| Allyl methacrylate crosspolymer | 0.5 |
| Hydroxypropylmethylcellulose | 15 |
| Titanium dioxide (Eusolex T2000) | 9 |
| Titanium dioxide (titanium dioxide T805) | 5 |
| Behenoxy dimethicone (Abil Wax 2440) | 2 |
| Caprylic/capric triglyceride | 10 |
| Mineral oil | 9 |
| Butylene glycol caprylate/caprate | 9 |
| Dicaprylyl ether (Cetiol OE) | 3.5 |
| UVASORB ® K2A | 3 |
| Ethylhexyltriazone | 6 |
| Diethylhexylbutamidotriazone | 5 |
| Phenyldibenzimidazoletetrasulphonic | 3 |
| acid | |
| Phenylbenzimidazolesulphonic acid | 4 |
| Mannitol | 3 |
| Biosaccharide gel (Fucogel 1000) | 2 |
| Lactic acid | 0.5 |
| Polyquaternium 37 | 2 |
| NaOH 45% strength solution in water | 3 |
| Perfume | 1 |
| Water | ad 100 |

Preparation 4: Photoprotective spray

| | % by wt. |
|---|---|
| Quarternium-18 hectorite | 1 |
| Ethylene/propylene/styrene/copolymer | 3 |
| Allyl methacrylate crosspolymer | 0.5 |
| Hydroxypropylmethylcellulose | 2 |
| Polyvinyl alcohol | 5 |
| Polyvinylpyrrolidone | 5 |
| Carbomer (Carbopol 981) | 1 |
| Polyacrylate | 2 |
| Hydroxypropylmethylcellulose | 5 |
| Silica (Aerosil R972) | 2 |
| C₁₆₋₃₈-Alkylhydroxystearoyl stearate (Kester wax | 2 |
| K80P) | |
| Caprylic/capric triglyceride | 5 |
| Octyldodecanol | 5 |
| Mineral oil | 5 |
| Butylene glycol caprylate/caprate | 6 |
| Ubiquinone | 1 |
| Butylmethoxydibenzoylmethane | 5 |
| 3-(4-Methylbenzylidene)camphor | 3 |
| Ethylhexyl methoxycinnamate | 9 |
| Methylene bisbenzotriazolyl tetramethylbutylphenol | 8 |
| Phenylbenzimidazole sulphonic acid | 8 |
| Diethylhexylbutamidotriazone | 4 |
| (UVASORB HEB) | |
| Sodium maize starch n-octenyl succinate | 1 |
| Chitosan | 2 |
| NaCl | 3 |
| C₁₀₋₃₀-Alkyl acrylate crosspolymer (Pemulen TR1) | 2 |
| Methylhydroxyethylcellulose | 4 |
| Water | ad 100 |

Preparation 5: Photoprotective preparation

| | % by wt. |
|---|---|
| Ethylcellulose | 5 |
| Polyvinyl alcohol | 4 |
| Hydroxyethylcellulose | 5 |
| Dimethicone/polysilicone-11 | 2 |
| Polyquaternium 37 | 2 |
| Hydroxypropylmethylcellulose | 5 |
| Titanium dioxide (Eusolex T2000) | 10 |
| Silica (Aerosil R972) | 2 |
| Sodium maize starch n-octenyl succinate | 1 |
| C₂₀₋₄₀-Alkyl stearate (Kester wax K82) | 2 |
| Polyisobutene (Rewopal PIB 1000) | 4 |
| Caprylic/capric triglyceride | 5 |
| Mineral oil | 5 |
| PVP/hexadecene copolymer | 4 |
| Acetylated glycol stearate + tristearin | 2 |
| Tocopheryl acetate | 1 |
| UVINUL ® A Plus | 5 |
| Ethylhexyl methoxycinnamate | |
| Octocrylene | 8 |
| Phenyldibenzimidazoletetrasulphonic acid | 7 |
| Homosalate | 3 |
| Ethylhexyl salicylate | 4 |
| Tapioca starch | 4 |
| Hyaluronic acid | 4 |
| Magnesium sulphate | 3 |
| C₁₀₋₃₀-Alkyl acrylate crosspolymer (Pemulen | 2 |
| TR1) | |
| Water | ad 100 |

Auxiliary mixtures for preparations 1 to 5

| Data in % by wt. | 1 | 2 | 3 | 4 | 5 |
|---|---|---|---|---|---|
| Microcrystalline cellulose (Avicel PH | 15 | | | | 17.5 |
| 102) | | | | | |
| Crosslinked sodium | 2 | | | 2 | |
| carboxymethylcellulose (Ac-Di-Sol) | | | | | |
| Crosslinked polyvinylpyrrolidone | | | 5 | | |
| Sodium hydrogencarbonate | | 2 | | 1 | |
| Finely divided silica (Sipernat 220) | 1 | | 2 | | |
| Citric acid | | 4.6 | | 2.3 | |
| Milk sugar | 3 | 5 | 4 | | |
| Sucrose | | 6 | | 4 | |
| Gelatin | | | 1.5 | | |
| Starch | | 1 | | | |
| Glycerol | | 3 | | | |
| Sorbitol | 2 | | 3 | | |
| Starch | | | | | |
| Aerosil 200 | | | 1.5 | 1 | |
| Stearic acid | 1.5 | | 1 | | |
| Magnesium stearate | | 2 | | 1 | |
| Talc | 1 | | 2 | | |
| Carbomer (Carbopol 981) | | | | 3 | |
| Polyvinylpyrrolidone | | | | | 4 |
| Xanthan gum | 3 | | | | |
| Hydroxyethylcellulose | | | 2.5 | | |
| C₁₀₋₃₀-Alkyl acrylate crosspolymer | | 4 | | | |
| (Pemulen TR1) | | | | | |
| Magnesium aluminium silicate | | | | 1 | |
| (Veegum K) | | | | | |
| Alcohol | | 20 | 15 | 10 | |

1. A cosmetic or dermatological preparation obtainable by drying an oil-in-water emulsion comprising:
a) a lipid phase in a concentration of from 10 to 85% by weight, based on the total weight of the preparation before drying and having one or more oil-soluble polymers in a total concentration of from 0.1 to 50% by weight, based on the total weight of the preparation before drying;
b) an aqueous phase having one or more water-soluble polymers in a total concentration of from 1 to 80% by weight, based on the total weight of the preparation before drying; and
c) one or more UV photoprotective filters.

2. A cosmetic or dermatological preparation according to claim 1, further comprising one or more active ingredients, auxiliaries, or additives that are present in the preparation from 0.1 to 20% by weight, based on the weight of the dried preparation.

3. A cosmetic or dermatological preparation according to claim 1, further comprising granulation auxiliaries, tablet auxiliaries, or combinations thereof.

4. A cosmetic or dermatological preparation according to claim 3, wherein the granulation or tablet auxiliaries are selected from the group consisting of tablet filling auxiliaries, tablet compacting auxiliaries, tablet mould release agents, tablet binders, tablet fillers, tablet glidants, tablet lubricants, tablet flow agents, tablet disintegrants, and tablet coatings.

5. A cosmetic or dermatological preparation according to claim 4, wherein the granulation or tablet auxiliaries include one or more auxiliaries selected from the group consisting of starch derivatives, cellulose derivatives, highly disperse silicon dioxides, carbonates, hydrogen carbonates, sodium hydrogen carbonate, potassium hydrogen carbonate, acids that are solid at room temperature, citric acid, ascorbic acid, lactic acid, tartaric acid, and crosslinked polyvinylpyrrolidone.

6. A cosmetic or dermatological preparation according to claim 3, wherein the granulation or tablet auxiliaries are present in a concentration of from 0.5 to 35% by weight, based on the total weight of the preparation.

7. A cosmetic or dermatological preparation according to claim 1, wherein the preparation is in the form of a sunscreen powder.

8. A cosmetic or dermatological preparation according to claim 1, wherein the preparation is in the form of a cosmetically decorative powder.

9. A dried oil-in-water emulsion concentrate comprising:
a lipid phase comprising a plurality of oil droplets;
one or more oil-soluble polymers disposed within the plurality of oil droplets;
an aqueous phase that is interdispersed between the oil droplets and is present in an amount of less than 8 percent by weight, based on the total weight of the concentrate; and
one or more water-soluble polymers that are present in the aqueous phase so that the oil droplets are prevented from coalescing.

10. A dried oil-in-water emulsion concentrate according to claim 9, further comprising one or more UV photoprotective filters.

11. A dried oil-in-water emulsion concentrate according to claim 9, wherein the concentrate is in the form of a tablet.

12. A dried oil-in-water emulsion concentrate according to claim 11, wherein the concentrate includes one or more tablet auxiliaries selected from the group consisting of starch derivatives, cellulose derivatives, highly disperse silicon dioxides, carbonates, hydrogen carbonates, sodium hydrogen carbonate, potassium hydrogen carbonate, citric acid, ascorbic acid, lactic acid, tartaric acid, and crosslinked polyvinylpyrrolidone,

13. A dried oil-in-water emulsion concentrate according to claim 11, further comprising one or more tablet auxiliaries selected from the group consisting of carbonates, hydrogen carbonates, and one or more acids that are solid at room temperature.

14. A dried oil-in-water emulsion concentrate according to claim 9, wherein the concentrate further comprises from 0.5 to 15% by weight hydrogen carbonate, and from 0.5 to 15% by weight acids that are solid at room temperature, based on the total weight of the concentrate.

15. A process of preparing a cosmetic or dermatological concentrate comprising:
providing an oil-in-water emulsion comprising:
a. lipid phase in a concentration of from 10 to 85% by weight, based on the total weight of the emulsion and having one or more oil-soluble polymers in a total concentration of from 0.1 to 50% by weight, based on the total weight of the emulsion, and
b. an aqueous phase having one or more water-soluble polymers in a total concentration of from 1 to 80% by weight, based on the total weight of the emulsion, and
c. one or more UV photoprotective filters;
drying the emulsion to produce a concentrate wherein the volatile constituents have been removed.

16. A process according to claim 15, wherein the step of drying further comprises using drum drying, tunnel drying, spray-drying, freeze-drying, or combinations thereof.

17. A process according to claim 15, further comprising mixing the concentrate in a mixing device with one or more active ingredients, additives, or auxiliaries that are water-soluble or readily volatile.

18. A process according to claim 15, further comprising pressing the concentrate into a compact shaped body and mixing the compact shaped body with water to form a re-emusified cosmetic preparation.

19. A process according to claim 15, further comprising mixing the concentrate with one or more tablet auxiliaries in a mixing device.

20. A process according to claim 15, further comprising mixing the concentrate with water to re-emulsify the concentrate under the action of motive forces.

21. A process according to claim 20, wherein the motive forces arise by shaking, stirring, centrifuging, or gases produced in situ.

22. A process according to claim 15, further comprising the step of pressing the concentrate in a press to produce one or more tablets.

23. A process according to claim 15, further comprising the step of granulating the concentrate using a granulation device.
